(19) United States
(12) Patent Application Publication
(10) Pub. No.: US 2026/0199120 A1
(43) Pub. Date: Jul. 16, 2026

(54) SHOULDER POSITIONING DEVICES AND RELATED METHODS

(71) Applicant: THE JOHNS HOPKINS UNIVERSITY, Baltimore, MD (US)

(72) Inventors: Kelsey MAAS, Baltimore, MD (US); Preeti RAGHAVAN, Baltimore, MD (US); Sandra DELUZIO, Severna Park, MD (US); Kelly JORDAN, Towson, MD (US); Julia KLAG, Baltimore, MD (US); Catharine SCOTT, Stewartstown, PA (US); Jeet Ketan ACHARYA, Baltimore, MD (US)

(21) Appl. No.: 19/127,855

(22) PCT Filed: Nov. 13, 2023
(86) PCT No.: PCT/US2023/037174

§ 371 Date: May 7, 2025

Related U.S. Application Data
(60) Provisional application No. 63/426,278, filed on Nov. 17, 2022.

(51) Int. Cl. A61F 5/37

(52) U.S. Cl. CPC A61F 5/3753

(57) ABSTRACT

Provided herein are shoulder positioning devices that include a base frame and an arm support structure. The base frame includes a base platform comprising a sliding mechanism operably connected to the base frame in which the sliding mechanism comprises a slider element that is configured to selectively move radially along at least one radius. The base frame also includes an attachment mechanism operably connected or connectable to the base frame in which the attachment mechanism is configured to reversibly attach the base frame to a subject. The arm support structure is operably connected or connectable to the slider element of the sliding mechanism and is configured to receive and support at least part of an arm of the subject. The arm support structure is also configured to be selectively rotated about at least one axis. Additional shoulder positioning devices and related methods are also provided.

### CROSS-REFERENCE TO RELATED APPLICATIONS

This application is the national stage entry of International Patent Application No. PCT/US2023/037174, filed on Nov. 13, 2023, and published as WO 2024/107391 A1 on May 23, 2024, which claims the benefit of U.S. Provisional Patent Application Ser. No. 63/426,278, filed on Nov. 17, 2022, which are hereby incorporated by reference herein in their entireties.

### BACKGROUND

Hemiplegic shoulder pain is one of the most common complications after stroke and can be associated with loss of shoulder range of motion. Weakness and immobility due to stroke may lead to the anterior chest muscles and the surrounding soft tissue being maintained in a shortened position which initially cause muscle stiffness and loss of range of motion and eventually lead to permanent changes in the muscle, referred to as contractures, which limit activities of daily living, participation in therapy, and the possibility of recovery of arm movement. Limitation in shoulder external rotation, shoulder abduction, and shoulder flexion are commonly documented after stroke.

Shoulder range of motion and muscle length may be preserved, and the progression to contracture potentially slowed by positioning the shoulder in comfortable external rotation for a minimum of 30 minutes a day. However, this position is difficult to achieve and maintain without a specific positioning device in people with hemiplegia. Currently, there is no device on the market to facilitate maintaining a patient's affected shoulder in a position of maximum comfortable external rotation. Previous studies have used pillows and sandbags while the patient was in bed to achieve the desired position. The goal for a patient in rehabilitation is to be out of bed as much as possible, and there are presently no positioning devices available to achieve and maintain shoulder external rotation in sitting and lying positions.

Accordingly, there is a need for positioning devices that maintain an affected shoulder of a given patient in a position of maximum comfortable external rotation.

### SUMMARY

The present disclosure relates, in certain aspects, to shoulder positioning devices and methods of use in maintaining an affected shoulder of a patient or other subject in a position of maximum comfortable external rotation and in effecting abduction of an arm of a subject (e.g., for stroke-affected patients during rehabilitation or the like), among other attributes. These and other aspects will be apparent upon a complete review of the present disclosure, including the accompanying figures.

In one aspect, the present disclosure provides a shoulder positioning device that includes a base frame comprising a base platform comprising first and second surfaces and a sliding mechanism operably connected to the base frame, wherein the sliding mechanism comprises a slider element that is configured to selectively move radially along at least one radius, and an attachment mechanism operably connected or connectable to the base frame, wherein the attachment mechanism is configured to reversibly attach the base frame to a subject when the subject uses the shoulder positioning device. The shoulder positioning device also includes an arm support structure operably connected or connectable to the slider element of the sliding mechanism, wherein the arm support structure is configured to receive and support at least part of an arm of the subject when the subject uses the shoulder positioning device and wherein the arm support structure is configured to be selectively rotated about at least one axis. In some embodiments, the shoulder positioning device further comprises a covering structure disposed over at least a portion of the base frame.

In some embodiments, the shoulder positioning device further comprises a first height adjustment mechanism operably connected to the base platform, wherein a height of the first height adjustment mechanism is configured to be selectively adjusted relative to the first surface of the base platform. In some embodiments, the shoulder positioning device further comprises a second height adjustment mechanism operably connected to the base platform, wherein a height of the second height adjustment mechanism is configured to be selectively adjusted relative to the second surface of the base platform. In some embodiments, the shoulder positioning device further comprises a main platform comprising first and second surfaces, wherein the main platform is operably connected to the first height adjustment mechanism. In some embodiments, the first height adjustment mechanism and/or the second height adjustment mechanism are configured to effect abduction of an arm of a subject when the arm support structure receives the arm of the subject.

In some embodiments, the first height adjustment mechanism is operably connected to the first surface of the base platform. In some embodiments, the height of the first height adjustment mechanism is configured to be selectively adjusted between about 10 mm and about 300 mm relative to the first surface of the base platform. In some embodiments, the first height adjustment mechanism comprises a set of telescopic legs. In some embodiments, the set of telescopic legs comprises one, two, three, four, five, or more telescopic legs. In some embodiments, a given telescopic leg of the first height adjustment mechanism comprises two, three, four, five, six, seven, eight, nine, or more telescoping sections.

In some embodiments, the second height adjustment mechanism is operably connected to the second surface of the base platform. In some embodiments, the height of the second height adjustment mechanism is configured to be selectively adjusted between about 10 mm and about 300 mm relative to the second surface of the base platform. In some embodiments, the second height adjustment mechanism comprises a set of telescopic legs. In some embodiments, the set of telescopic legs comprises one, two, three, four, five, or more telescopic legs. In some embodiments, a given telescopic leg of the second height adjustment mechanism comprises two, three, four, five, six, seven, eight, nine, or more telescoping sections.

In some embodiments, the sliding mechanism is operably connected to the first surface of the main platform. In some embodiments, the slider element comprises a hinge structure. In some embodiments, an orifice is disposed at least partially through the hinge structure, wherein a plurality of slots is disposed through a wall of the hinge structure, which plurality of slots communicate with the orifice, wherein a cylindrical tube element extends from the arm support structure, wherein at least a portion of the cylindrical tube element rotatably fits within the orifice, wherein at least one press button element is resiliently coupled to the cylindrical tube element, and wherein at least a portion of the press button element is configured to be reversibly disposed through any one of the plurality of slots such that the arm support structure is selectively rotatable about the axis. In some embodiments, the plurality of slots comprises from about 10 to about 30 slots, wherein pairs of slots are disposed substantially diametrically opposite one another through the wall of the hinge structure. In some embodiments, two press button elements are resiliently coupled to the cylindrical tube element, which press button elements are disposed substantially diametrically opposite one another on a circumference of the cylindrical tube. In some embodiments, the cylindrical tube element extends from the arm support structure via a platform component. In some embodiments, the hinge structure comprises a substantially tubular shape.

In some embodiments, the plurality of slots provides angular adjustability to the arm support structure to effect external and/or internal rotation of the arm and a shoulder of the subject when the subject uses the shoulder positioning device. In some embodiments, the press button element is rotatable at least between a locked position and an unlocked position. In some embodiments, the press button element is resiliently coupled to the cylindrical tube element via at least one spring. In some embodiments, the sliding mechanism comprises a slider element guide along which the slider element selectively moves. In some embodiments, the slider element guide comprises a track element that extends along at least a portion of a length of the sliding mechanism and wherein the slider element comprises a track element receiving area that is configured to receive at least a segment of the track element. In some embodiments, the sliding mechanism comprises a cover structure. In some embodiments, the cover structure comprises at least one wall that limits movement of the slider element. In some embodiments, the cover structure comprises two, three, four, or more walls. In some embodiments, the sliding mechanism comprises a retaining mechanism that is configured to reversibly retain the slider element at a selected position along the radius. In some embodiments, the sliding mechanism is adjustable for use on subjects with different arm lengths. In some embodiments, the retaining mechanism comprises a body structure having a plurality of gripping surfaces. In some embodiments, the retaining mechanism comprises a knob element that is configured to reversibly move the body structure such that at least one of the gripping surfaces grips the slider element to reversibly retain the slider element at the selected position along the radius. In some embodiments, the knob element comprises a threaded cylindrical shaft component that is received by a corresponding threaded opening disposed through the wall of the cover structure, wherein the threaded cylindrical shaft component is configured to contact the body structure.

In some embodiments, the attachment mechanism comprises a vest that is configured to be worn around a torso portion of the subject. In some embodiments, the attachment mechanism comprises a belt that is configured to be worn around a torso portion of the subject. In some embodiments, the torso portion of the subject comprise a waist area of the subject. In some embodiments, the attachment mechanism is operably connected or connectable to the base platform.

In some embodiments, the arm support structure is configured to substantially prevent an elbow of the subject from adducting when the subject uses the shoulder positioning device. In some embodiments, the arm support structure is configured to adjust a degree of abduction of an upper arm of the subject relative to the torso of the subject when the subject uses the shoulder positioning device. In some embodiments, the arm support structure comprises a tapered trough structure having an opening that is configured to receive at least the part of the arm of the subject when the subject uses the shoulder positioning device. In some embodiments, the arm support structure is configured to be selectively rotated about the axis to effect external and/or internal rotation of the arm and a shoulder of the subject when the subject uses the shoulder positioning device. In some embodiments, the arm support structure is configured to be selectively rotated about the axis to effect measurable amounts of external and/or internal rotation of the arm and a shoulder of the subject when the subject uses the shoulder positioning device. In some embodiments, the arm support structure comprises one or more reversibly attachable retaining straps that are configured to retain at least a portion of the arm of the subject at least partially within the arm support structure when the subject uses the shoulder positioning device.

In another aspect, the present disclosure provides a shoulder positioning device that includes a base frame comprising a base platform comprising first and second surfaces, a first height adjustment mechanism operably connected to the base platform, wherein a height of the first height adjustment mechanism is configured to be selectively adjusted relative to the first surface of the base platform, a second height adjustment mechanism operably connected to the base platform, wherein a height of the second height adjustment mechanism is configured to be selectively adjusted relative to the second surface of the base platform, a main platform comprising first and second surfaces, wherein the main platform is operably connected to the first height adjustment mechanism, a sliding mechanism operably connected to the main platform, wherein the sliding mechanism comprises a slider element that is configured to selectively move radially along at least one radius, and an attachment mechanism operably connected or connectable to the base frame, wherein the attachment mechanism is configured to reversibly attach the base frame to a subject when the subject uses the shoulder positioning device. The shoulder positioning device also includes an arm support structure operably connected or connectable to the slider element of the sliding mechanism, wherein the arm support structure is configured to receive and support at least part of an arm of the subject when the subject uses the shoulder positioning device and wherein the arm support structure is configured to be selectively rotated about at least one axis.

In another aspect, the present disclosure provides a method of positioning a shoulder of a subject. The method includes attaching a shoulder positioning device to the subject using an attachment mechanism of the shoulder positioning device, wherein the shoulder positioning device comprises a base frame comprising a base platform comprising first and second surfaces, a sliding mechanism operably connected to the base frame, wherein the sliding mechanism comprises a slider element that is configured to selectively move radially along at least one radius, and the attachment mechanism operably connected to the base frame, wherein the attachment mechanism is configured to reversibly attach the base frame to the subject. The shoulder positioning device also includes an arm support structure operably connected to the slider element of the sliding mechanism, wherein the arm support structure is configured to receive and support at least part of an arm of the subject and wherein the arm support structure is configured to be selectively rotated about at least one axis. The method also includes placing an arm of the subject in the arm support structure of the shoulder positioning device, moving the slider element of the sliding mechanism radially along the radius to a selected radial position relative to the subject, and rotating the arm support structure axially about the axis to a selected axial position relative to the subject, thereby positioning the shoulder of the subject.

In some embodiments, the shoulder positioning device further comprises a first height adjustment mechanism operably connected to the base platform, wherein a height of the first height adjustment mechanism is configured to be selectively adjusted relative to the first surface of the base platform, and wherein the method further comprises adjusting the height of the first height adjustment mechanism relative to the first surface of the base platform. In some embodiments, the shoulder positioning device further comprises a second height adjustment mechanism operably connected to the base platform, wherein a height of the second height adjustment mechanism is configured to be selectively adjusted relative to the second surface of the base platform, wherein the shoulder positioning device further comprises a main platform comprising first and second surfaces, wherein the main platform is operably connected to the first height adjustment mechanism, wherein the sliding mechanism is operably connected to the first surface of the main platform, and wherein the method further comprises adjusting the height of the second height adjustment mechanism relative to the second surface of the base platform.

### BRIEF DESCRIPTION OF THE DRAWINGS

The accompanying drawings, which are incorporated in and constitute a part of this specification, illustrate certain embodiments, and together with the written description, serve to explain certain principles of the devices and related methods disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1A schematically depicts a shoulder positioning device from a perspective view according to an exemplary embodiment.

FIG. 1B schematically depicts aspects of the shoulder positioning device from FIG. 1A from a perspective view.

FIG. 1C schematically depicts the shoulder positioning device from FIG. 1A from a top view.

FIG. 1D schematically depicts a sliding mechanism of the shoulder positioning device from FIG. 1A from a perspective view.

FIG. 1E schematically depicts the arm support structure of the shoulder positioning device from FIG. 1A from a perspective view.

FIG. 1F schematically depicts the arm support structure from FIG. 1E from a side view.

FIG. 1G schematically depicts a spring that is used to resiliently couple a press button element to a cylindrical tube element in the sliding mechanism from FIG. 1D from a side view.

FIG. 1H schematically depicts a press button element of the sliding mechanism from FIG. 1D from a perspective view.

FIG. 1I schematically depicts a press button element reversibly disposed through one of a plurality of slots of a hinge structure of the sliding mechanism from FIG. 1D from a side view.

FIG. 1J schematically depicts the shoulder positioning device from FIG. 1A from a top view.

FIG. 1K schematically depicts the shoulder positioning device from FIG. 1A from a side view.

FIG. 2A schematically depicts a shoulder positioning device from a perspective view according to an exemplary embodiment.

FIG. 2B schematically depicts aspects of the shoulder positioning device from FIG. 2A from a perspective view.

FIG. 2C schematically depicts the arm support structure of the shoulder positioning device from FIG. 2A from a perspective view.

FIG. 3A schematically depicts a shoulder positioning device from a perspective view according to an exemplary embodiment.

FIG. 3B schematically depicts the shoulder positioning device from FIG. 3A from another perspective view.

FIG. 4 schematically depicts a shoulder positioning device from a perspective view according to an exemplary embodiment.

FIG. 5 schematically depicts a shoulder positioning device from a perspective view according to an exemplary embodiment.

FIG. 6 is an image of a subject seated in a wheelchair in which the subject's arm is positioned in a shoulder positioning device according to an exemplary embodiment.

FIG. 7A is an image that depicts a shoulder positioning device having an attachment mechanism that comprises a vest that is configured to be worn around a torso portion of a subject from a front view according to an exemplary embodiment.

FIG. 7B is an image that depicts the shoulder positioning device from FIG. 7A from a back view.

FIG. 8 is a flow chart that schematically depicts exemplary method steps according to some aspects disclosed herein.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, devices, kits, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Communicate: As used herein, “communicate” refers to the direct or indirect transfer or transmission, and/or capability of directly or indirectly transferring or transmitting, something at least from one area to another area.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

### DETAILED DESCRIPTION

The present disclosure provides shoulder positioning devices and related methods of use in maintaining a shoulder of a subject (e.g., a stroke patient, etc.) in a position that maximizes the comfort level of external rotation of that shoulder. Among other attributes, the shoulder positioning devices and related methods of the present disclosure also effect abduction of a subject's arm positioned in the devices. These and other features and aspects will be apparent upon a complete review of the present disclosure, including the accompanying figures.

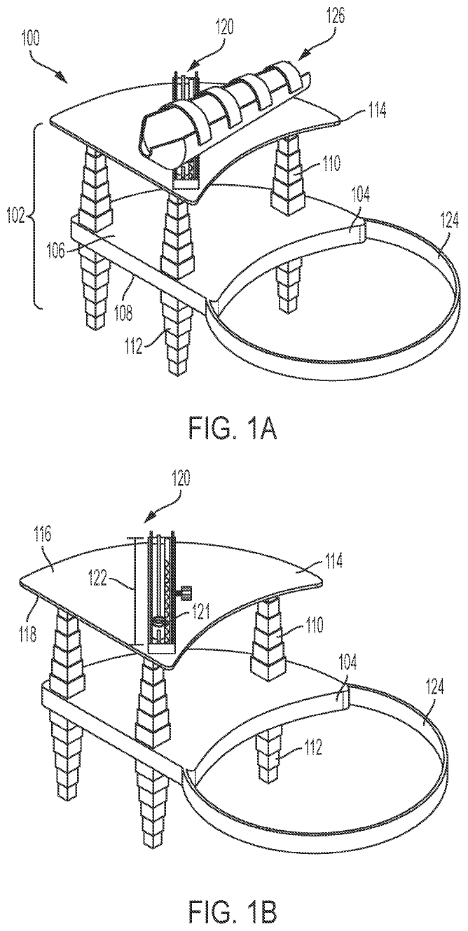

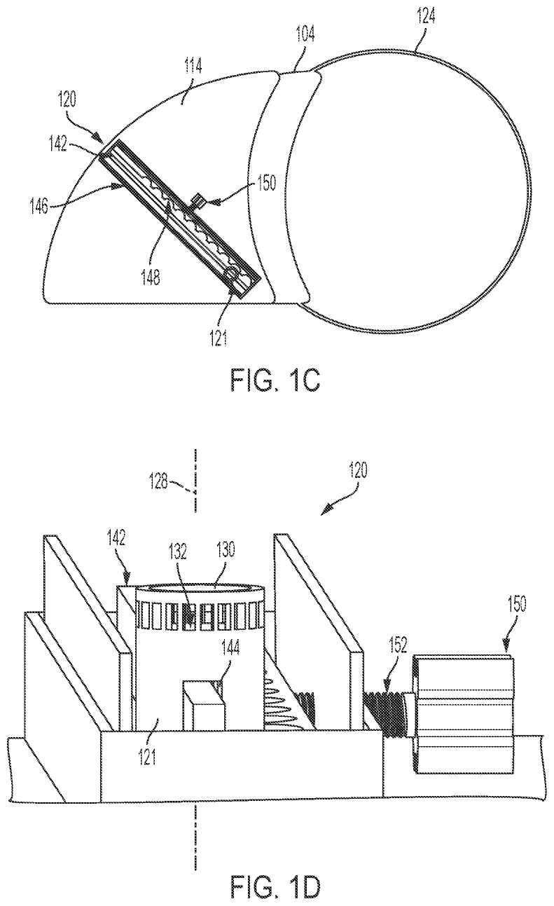

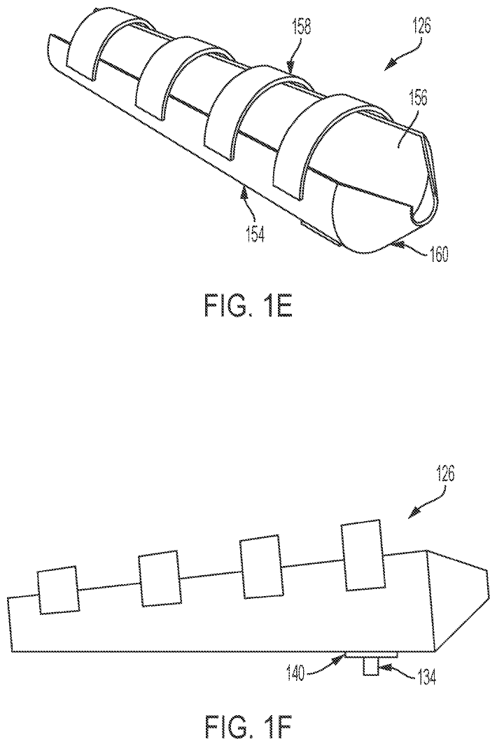

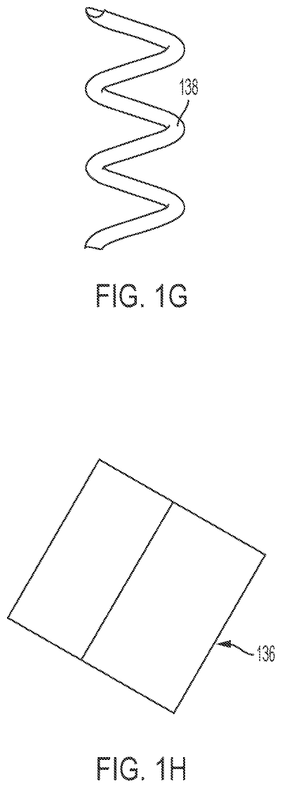

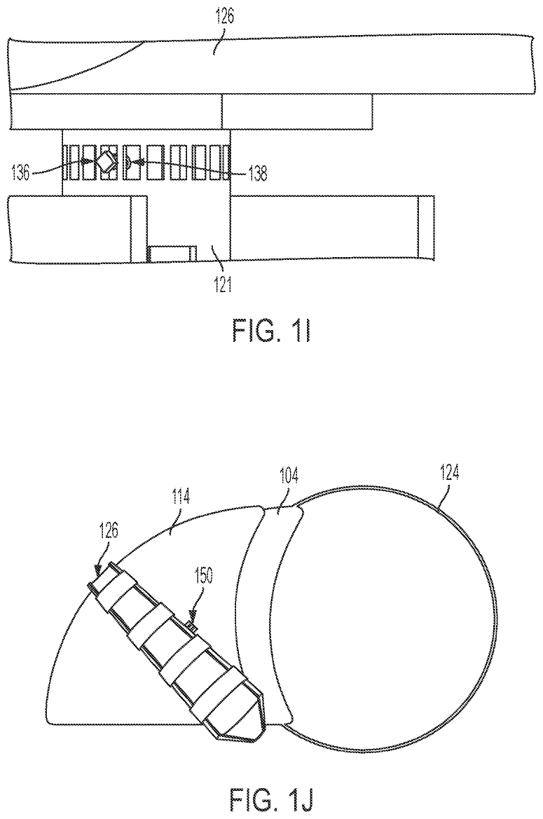

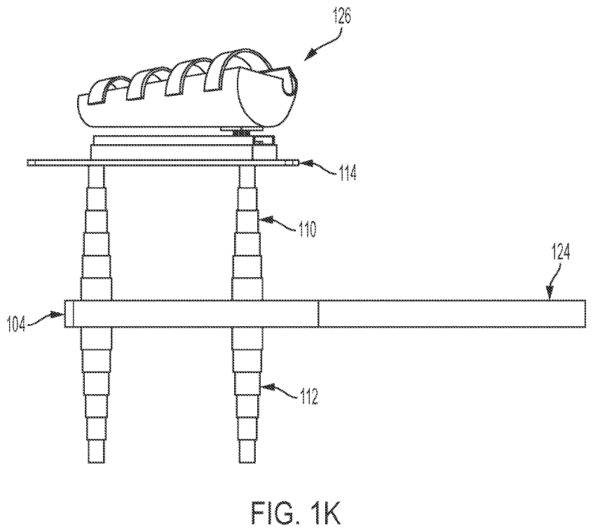

To illustrate, FIGS. 1A-1K schematically depicts a shoulder positioning device or aspects thereof from various views according to an exemplary embodiment. As shown, shoulder positioning device 100 includes base frame 102 that includes base platform 104, which includes first surface 106 and second surface 108. Shoulder positioning device 100 also includes first height adjustment mechanism 110 (shown as including a set of three telescopic legs in this embodiment) operably connected to base platform 104 (via first surface 106) in which a height of first height adjustment mechanism 110 is configured to be selectively adjusted relative to first surface 106 of base platform 104. Shoulder positioning device 100 also includes second height adjustment mechanism 112 (shown as including a set of three telescopic legs in this embodiment) operably connected to base platform 104 (via second surface 108) in which a height of second height adjustment mechanism 112 is configured to be selectively adjusted relative to second surface 108 of base platform 104. As also shown, base frame 102 additionally includes main platform 114 comprising first surface 116 and second surface 118 in which main platform 114 is operably connected to first height adjustment mechanism 110 (via second surface 118). Base frame 102 also includes sliding mechanism 120 operably connected to main platform 114 (via first surface 116) in which sliding mechanism 120 comprises slider element 121 that is configured to selectively move radially along radius 122. In addition, base frame 102 also includes attachment mechanism 124 (shown as a belt) operably connected or connectable to base frame 104 in which attachment mechanism 124 is configured to reversibly attach base frame 104 to a subject when the subject uses shoulder positioning device 100. As also shown, shoulder positioning device 100 also includes arm support structure 126 operably connected or connectable to slider element 121 of sliding mechanism 120 in which arm support structure 126 is configured to receive and support at least part of an arm of a subject when the subject uses shoulder positioning device 100 and in which arm support structure 126 is configured to be selectively rotated about axis 128 (shown as a dashed line).

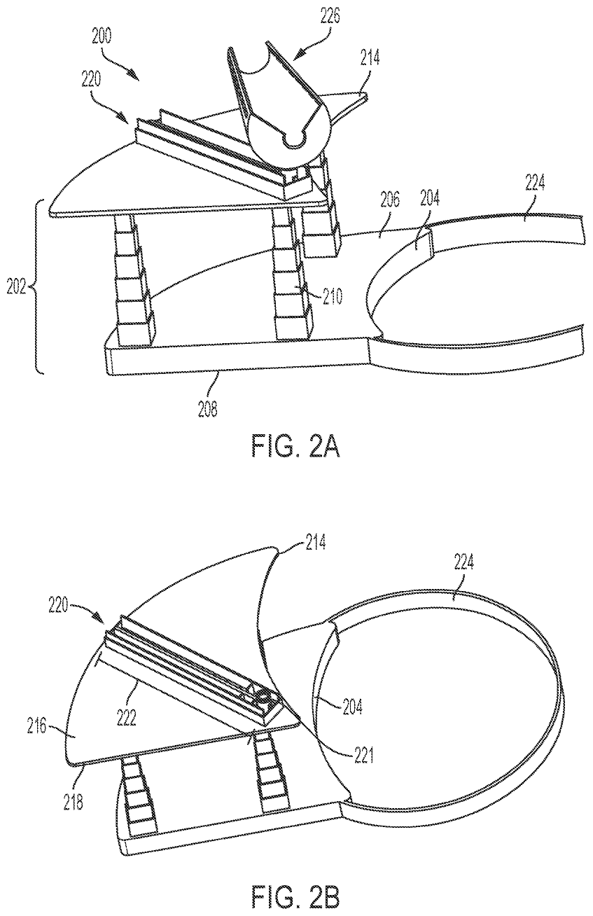

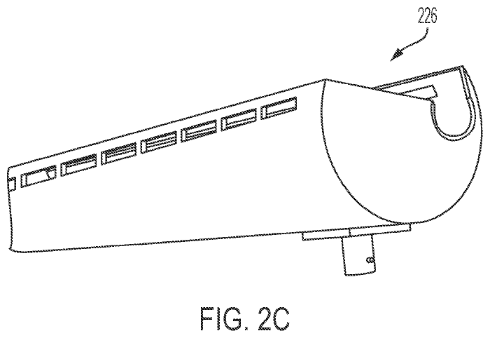

In some embodiments, base frames of shoulder positioning devices include only a first height adjustment mechanism or a second height adjustment mechanism, but not both. An example of such an embodiment in shown in FIGS. 2A-2C. As shown, shoulder positioning device 200 includes base frame 202 that includes base frame 204, which includes first surface 206 and second surface 208. Shoulder positioning device 200 also includes first height adjustment mechanism 210 (shown as including a set of three telescopic legs in this embodiment) operably connected to base platform 204 (via first surface 206) in which a height of first height adjustment mechanism 210 is configured to be selectively adjusted relative to first surface 206 of base platform 204. Shoulder positioning device 200 does not include a second height adjustment mechanism, unlike shoulder positioning device 100. As also shown, base frame 202 additionally includes main platform 214 comprising first surface 216 and second surface 218 in which main platform 214 is operably connected to first height adjustment mechanism 210 (via second surface 218). Base frame 202 also includes sliding mechanism 220 operably connected to main platform 214 (via first surface 216) in which sliding mechanism 220 comprises slider element 221 that is configured to selectively move radially along radius 222. In addition, base frame 202 also includes attachment mechanism 224 operably connected or connectable to base platform 204 in which attachment mechanism 224 is configured to reversibly attach base frame 204 to a subject when the subject uses shoulder positioning device 200. As also shown, shoulder positioning device 200 also includes arm support structure 226 operably connected or connectable to slider element 221 of sliding mechanism 220 in which arm support structure 226 is configured to receive and support at least part of an arm of a subject when the subject uses shoulder positioning device 200 and in which arm support structure 226 is configured to be selectively rotated about an axis (similar to axis 128 as referenced herein).

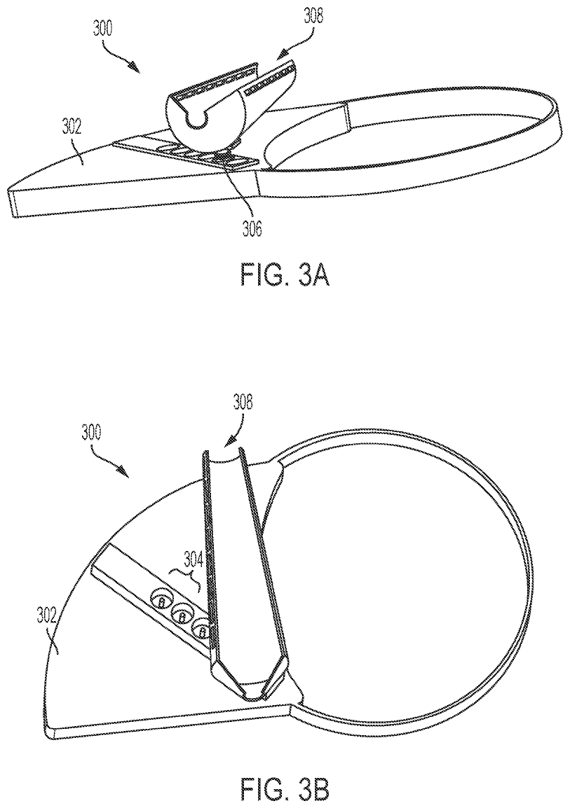

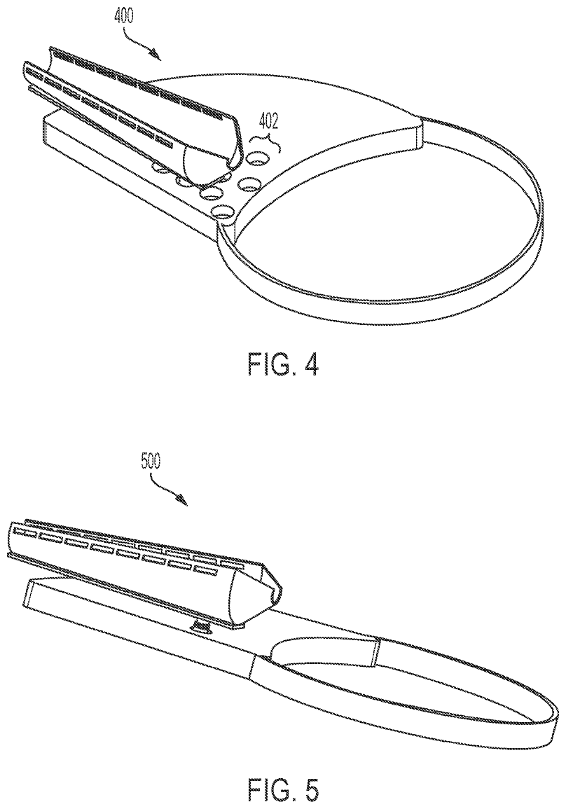

In some embodiments, base frames of shoulder positioning devices include do not include any height adjustment mechanisms. Examples of these embodiments are schematically shown in FIGS. 3A, 3B, 4, and 5. As shown in FIGS. 3A and 3B, for example, shoulder positioning device 300 includes a base frame that includes base platform 302. Although not shown, a sliding mechanism, as described herein, is optionally operably connected to base platform 302 in which the sliding mechanism comprises a slider element that is configured to selectively move radially along at least one radius. In the embodiment shown, however, base platform 302 includes linear array of circular slots 304 that are configured to reversibly receive corresponding circular projection 306 of arm support structure 308. Shoulder positioning device 300 also includes attachment mechanism 308 operably connected or connectable to base platform 302. Various attachment mechanisms are described further herein. In the embodiment schematically shown in FIG. 4, shoulder positioning device 400 is essentially the same as shoulder positioning device 300 aside from having dispersed array of circular slots 402, instead of linear array of circular slots 304. In contrast, in the embodiment schematically shown in FIG. 5, shoulder positioning device 500 is also essentially the same as shoulder positioning device 300, but lacks either a linear or a dispersed array of circular slots 402. Instead, the arm support structure of shoulder positioning device is rotatably or fixedly coupled to the base platform of shoulder positioning device 500 at a single position.

The first and second height adjustment mechanism include various embodiments. In some embodiments, for example, a shoulder positioning device includes a first height adjustment mechanism operably connected to the base platform in which a height of the first height adjustment mechanism is configured to be selectively adjusted relative to the first surface of the base platform. In some embodiments, a shoulder positioning device includes a second height adjustment mechanism operably connected to the base platform in which a height of the second height adjustment mechanism is configured to be selectively adjusted relative to the second surface of the base platform. In some embodiments, a shoulder positioning device includes a main platform comprising first and second surfaces in which the main platform is operably connected to the first height adjustment mechanism. In some embodiments, the first height adjustment mechanism is operably connected to the first surface of the base platform. In some embodiments, the second height adjustment mechanism is operably connected to the second surface of the base platform. In some embodiments, the height of the first height adjustment mechanism is configured to be selectively adjusted between about 10 mm and about 300 mm relative to the first surface of the base platform. In some embodiments, the height of the second height adjustment mechanism is configured to be selectively adjusted between about 10 mm and about 300 mm relative to the second surface of the base platform. In some embodiments, the first height adjustment mechanism and/or the second height adjustment mechanism comprises a set of telescopic legs. In some embodiments, the set of telescopic legs comprises one, two, three, four, five, or more telescopic legs. In some embodiments, a given telescopic leg of the first height adjustment mechanism comprises two, three, four, five, six, seven, eight, nine, or more telescoping sections. Typically, the first height adjustment mechanism and/or the second height adjustment mechanism are configured to effect abduction of an arm of a subject when the arm support structure receives the arm of the subject.

As described herein, sliding mechanisms or other coupling structures are operably connected to the first surface of the main platform or to the first surface of the base platform depending on the embodiment of the shoulder positioning device. In some embodiments, the slider element comprises a hinge structure, such as slider element 121. As shown, for example, in some embodiments, orifice 130 is disposed at least partially through the hinge structure (e.g., slider element 121). As also shown, plurality of slots 132 is disposed through a wall of the hinge structure, which plurality of slots 132 communicate with orifice 130. Plurality of slots 132 are used for providing angular adjustability for providing external rotation of the patient's paretic arm in some embodiments. In some embodiments, cylindrical tube element 134 extends from arm support structure 126 in which at least a portion of cylindrical tube element 134 rotatably fits within orifice 130. In some embodiments, press button element 136 (shown as a square-shaped element) is resiliently coupled (e.g., via spring 138) to cylindrical tube element 134. In some of these embodiments, press button element 136 can be rotated. For example, press button element 136 is typically rotatable at least between a locked position and an unlocked position. For locking, one of the sides of the square can be rotated about 45 degrees with respect to the ground. For unlocking, one of the sides of the square press button element 136 is rotated to be substantially parallel with the ground in these embodiments. At least a portion of press button element 136 is configured to be reversibly disposed through any one of plurality of slots 132 such that arm support structure 126 is selectively rotatable about axis 128. Press button element 136 (again, square-shaped in this embodiment) typically has a side length that is lesser than the a given slot width. However, the diagonal length of press button element 136 is generally more than the given slot width. Also, spring 138 which is attached to press button element 136 typically has a diameter that is lesser than the slot width. This allows press button element 136 to be rotated and pushed inside the given slot while rotating the arm of a patient. After the cylindrical tube is twisted so that press button element 136 is in the desired slot, press button element 136 can be released so that they spring out and can be rotated by about 45 degrees, as mentioned above, to lock it again in a new position.

In some embodiments, the plurality of slots (e.g., plurality of slots 132) comprises from about 10 to about 30 slots in which pairs of slots are disposed substantially diametrically opposite one another through the wall of the hinge structure. In some embodiments, two press button elements are resiliently coupled to the cylindrical tube element. These press button elements are disposed substantially diametrically opposite one another on a circumference of the cylindrical tube. In some embodiments, the cylindrical tube element extends from the arm support structure via a platform component (e.g., platform component 140). In some embodiments, the hinge structure comprises a substantially tubular shape (e.g., hinge structure/slider element 121). Typically, the plurality of slots provides angular adjustability to the arm support structure to effect external and/or internal rotation of the arm and a shoulder of the subject when the subject uses the shoulder positioning device.

In some embodiments, sliding mechanism 120 comprises slider element guide 142 along which slider element 121 selectively moves. Slider element guide 142 comprises a track element that extends along at least a portion of a length of sliding mechanism 120. Slider element 121 comprises track element receiving area 144 that is configured to receive at least a segment of the track element. In some embodiments, sliding mechanism 120 comprises a cover structure. The cover structure comprises at least one wall 146 that limits movement of slider element 121. The cover structure generally comprises two, three, four, or more walls. As shown, sliding mechanism 120 includes four walls.

Sliding mechanism 120 comprises a retaining mechanism that is configured to reversibly retain slider element 121 at a selected position along radius 122. Sliding mechanism 120 is adjustable for use on subjects with different arm lengths. As shown, the retaining mechanism comprises body structure having a plurality of gripping surfaces 148. The retaining mechanism also includes knob element 150 that is configured to reversibly move the body structure 148 such that at least one of the gripping surfaces grips slider element 121 to reversibly retain slider element 121 at the selected position along radius 122. Knob element 150 typically comprises threaded cylindrical shaft component 152 that is received by a corresponding threaded opening disposed through the wall of the cover structure in which threaded cylindrical shaft component 152 is configured to contact the body structure.

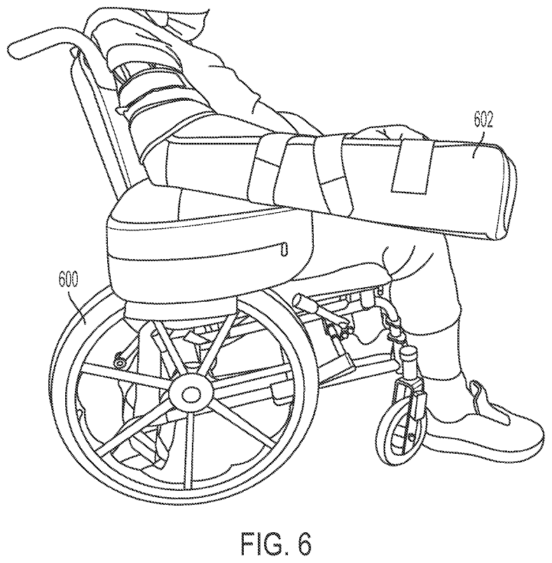

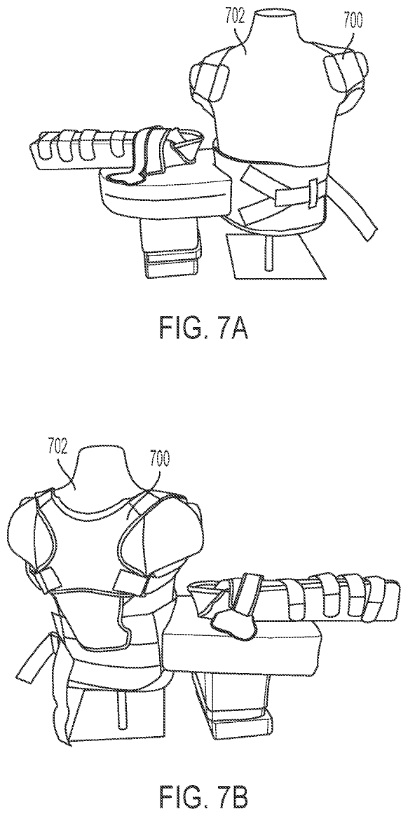

The attachment mechanisms of the shoulder positioning devices of the present disclosure include various embodiments. In some embodiments, for example, attachment mechanisms comprise a belt (e.g., belt 124) that is configured to be worn around a torso portion (e.g., a waist area) of the subject. In some embodiments, shoulder positioning devices are not attached to a given subject. In some of these embodiments, shoulder positioning devices are instead attached to another device, such as a wheelchair or other medical equipment used by a given subject. To illustrate, FIG. 6 is an image of a subject seated in wheelchair 600 in which the subject's arm is positioned in shoulder positioning device 602 according to an exemplary embodiment. In some embodiments, attachment mechanisms comprise a vest that is configured to be worn around a torso portion of the subject. An example of this embodiment is shown in FIGS. 7A and 7B, which are images that depict a shoulder positioning device having an attachment mechanism (shown as vest 700) that is configured to be worn around torso portion 702 (shoulders and waist area) of a subject according to an exemplary embodiment. In some embodiments, shoulder positioning devices also include a covering structure disposed over at least a portion of the base frame. Examples of covering structures are also shown in FIGS. 6, 7A, and 7B. In some embodiments, the cushions positioned below the arm support structures shown in FIGS. 6, 7A, and 7B are replaced with base frames as described herein. In some embodiments, the height of the arm support structures of the present disclosure is adjustable along the axis that the arm support structure is configured to be selectively rotated about.

The shoulder positioning devices of the present disclosure also include arm support structures having various embodiments. In some embodiments, arm support structures are configured to substantially prevent an elbow of the subject from adducting when the subject uses the shoulder positioning device. Typically, a given arm support structure is configured to adjust a degree of abduction of an upper arm of the subject relative to the torso of the subject when the subject uses the shoulder positioning device. As shown, arm support structure 126 comprises tapered trough structure 154 having opening 156 that is configured to receive at least the part of the arm of the subject when the subject uses shoulder positioning device 100. As described herein, arm support structure 126 is configured to be selectively rotated about axis 128 to effect external and/or internal rotation (e.g., measurable amounts of external and/or internal rotation) of the arm and shoulder of a subject when the subject uses shoulder positioning device 100. Arm support structure 126 comprises reversibly attachable retaining straps 158 (e.g., VELCRO® straps) that are configured to retain at least a portion of the arm of a subject at least partially within arm support structure 126 when the subject uses shoulder positioning device 100. In some embodiments, arm support structure 126 includes extra elbow covering 160 to support and protect an elbow of a given subject during use of shoulder positioning device 100. The extra covering provided at the bigger end of the tapered tube supports the elbow and prevents the arm from receding towards the body. As stroke patients generally have the tendency of receding their arm towards their body's sagittal plane, this extra covering prevents the patients' elbow from adducting. The tube and VELCRO® straps prevent the forearm and the hand from receding towards their body's sagittal plane, thus providing rehabilitative assistance. As also shown, for example, in FIG. 1J, arm support structure 126 is sometimes rotated to the position shown directly above sliding mechanism 120 before rotating knob element 150 to move hinge structure/slider element 121 radially.

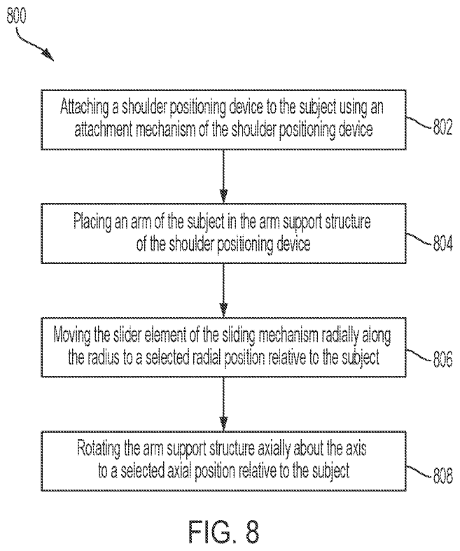

The present disclosure also provides different methods of using the shoulder positioning devices disclosed herein. To illustrate, FIG. 8 is a flow chart that schematically depicts exemplary method 800, which is a method of positioning a shoulder of a subject. As shown, method 800 includes attaching a shoulder positioning device as described herein to the subject using an attachment mechanism of the shoulder positioning device (step 802) and placing an arm of the subject in the arm support structure of the shoulder positioning device (step 804). Method 800 also includes moving the slider element of the sliding mechanism radially along the radius to a selected radial position relative to the subject (step 806) and rotating the arm support structure axially about the axis to a selected axial position relative to the subject (step 808). In some embodiments, the shoulder positioning device further includes a first height adjustment mechanism as described herein operably connected to the base platform, and in which method 800 further comprises adjusting the height of the first height adjustment mechanism relative to the first surface of the base platform. In some embodiments, the shoulder positioning device further comprises a second height adjustment mechanism as described herein operably connected to the base platform in addition to the first height adjustment mechanism and in which method 800 further comprises adjusting the height of the second height adjustment mechanism relative to the second surface of the base platform.

Shoulder positioning device components (e.g., base platforms, first height adjustment mechanisms, second height adjustment mechanisms, main platforms, sliding mechanisms, attachment mechanisms, arm support structures, etc.) are optionally formed by various fabrication techniques or combinations of such techniques including, e.g., extrusion, injection molding, cast molding, stamping, machining, embossing, engraving, etching (e.g., electrochemical etching, etc.), 3D printing, or other techniques. These and other suitable fabrication techniques are generally known in the art and described in, e.g., Molinari et al. (Eds.), Metal Cutting and High Speed Machining, Kluwer Academic Publishers (2002), Chung, Extrusion of Polymers: Theory and Practice, Hanser-Gardner Publications (2000), Altintas, Manufacturing Automation: Metal Cutting Mechanics, Machine Tool Vibrations, and CNC Design, Cambridge University Press (2000), Stephenson et al., Metal Cutting Theory and Practice, Marcel Dekker (1997), Fundamentals of Injection Molding, W. J. T. Associates (2000), Whelan, Injection Molding of Thermoplastics Materials, Vol. 2, Chapman & Hall (1991), Rosato, Injection Molding Handbook, 3rd Ed., Kluwer Academic Publishers (2000), Fisher, Extrusion of Plastics, Halsted Press (1976), and Redwood et al., The 3D Printing Handbook: Technologies, 1st Ed., Design and Applications, 3D Hubs (2017), which are each incorporated by reference. Exemplary materials optionally used to fabricate device components include, e.g., foam (e.g., polyurethane foam), metal (e.g., magnetic and/or non-magnetic), glass, wood, polymethylmethacrylate, polyethylene, polydimethylsiloxane, polyetheretherketone, polytetrafluoroethylene, polystyrene, polyvinylchloride, polypropylene, polysulfone, polymethylpentene, and polycarbonate, among many others. In some embodiments, following fabrication, device components are optionally further processed, e.g., by painting, coating surfaces with a hydrophilic coating or a hydrophobic coating, or the like.

Some further aspects are defined in the following clauses:

Clause 1: A shoulder positioning device, comprising: a base frame comprising: a base platform comprising first and second surfaces; a sliding mechanism operably connected to the base frame, wherein the sliding mechanism comprises a slider element that is configured to selectively move radially along at least one radius; and an attachment mechanism operably connected or connectable to the base frame, wherein the attachment mechanism is configured to reversibly attach the base frame to a subject when the subject uses the shoulder positioning device; and, an arm support structure operably connected or connectable to the slider element of the sliding mechanism, wherein the arm support structure is configured to receive and support at least part of an arm of the subject when the subject uses the shoulder positioning device and wherein the arm support structure is configured to be selectively rotated about at least one axis.

Clause 2: The shoulder positioning device of Clause 1, further comprising a first height adjustment mechanism operably connected to the base platform, wherein a height of the first height adjustment mechanism is configured to be selectively adjusted relative to the first surface of the base platform.

Clause 3: The shoulder positioning device of Clause 1 or Clause 2, further comprising a second height adjustment mechanism operably connected to the base platform, wherein a height of the second height adjustment mechanism is configured to be selectively adjusted relative to the second surface of the base platform.

Clause 4: The shoulder positioning device of any one of the preceding Clauses 1-3, further comprising a main platform comprising first and second surfaces, wherein the main platform is operably connected to the first height adjustment mechanism.

Clause 5: The shoulder positioning device of any one of the preceding Clauses 1-4, wherein the first height adjustment mechanism is operably connected to the first surface of the base platform.

Clause 6: The shoulder positioning device of any one of the preceding Clauses 1-5, wherein the height of the first height adjustment mechanism is configured to be selectively adjusted between about 10 mm and about 300 mm relative to the first surface of the base platform.

Clause 7: The shoulder positioning device of any one of the preceding Clauses 1-6, wherein the first height adjustment mechanism comprises a set of telescopic legs.

Clause 8: The shoulder positioning device of any one of the preceding Clauses 1-7, wherein the set of telescopic legs comprises one, two, three, four, five, or more telescopic legs.

Clause 9: The shoulder positioning device of any one of the preceding Clauses 1-8, wherein a given telescopic leg of the first height adjustment mechanism comprises two, three, four, five, six, seven, eight, nine, or more telescoping sections.

Clause 10: The shoulder positioning device of any one of the preceding Clauses 1-9, wherein the second height adjustment mechanism is operably connected to the second surface of the base platform.

Clause 11: The shoulder positioning device of any one of the preceding Clauses 1-10, wherein the height of the second height adjustment mechanism is configured to be selectively adjusted between about 10 mm and about 300 mm relative to the second surface of the base platform.

Clause 12: The shoulder positioning device of any one of the preceding Clauses 1-11, wherein the second height adjustment mechanism comprises a set of telescopic legs.

Clause 13: The shoulder positioning device of any one of the preceding Clauses 1-12, wherein the set of telescopic legs comprises one, two, three, four, five, or more telescopic legs.

Clause 14: The shoulder positioning device of any one of the preceding Clauses 1-13, wherein a given telescopic leg of the second height adjustment mechanism comprises two, three, four, five, six, seven, eight, nine, or more telescoping sections.

Clause 15: The shoulder positioning device of any one of the preceding Clauses 1-14, wherein the first height adjustment mechanism and/or the second height adjustment mechanism are configured to effect abduction of an arm of a subject when the arm support structure receives the arm of the subject.

Clause 16: The shoulder positioning device of any one of the preceding Clauses 1-15, wherein the sliding mechanism is operably connected to the first surface of the main platform.

Clause 17: The shoulder positioning device of any one of the preceding Clauses 1-16, wherein the slider element comprises a hinge structure.

Clause 18: The shoulder positioning device of any one of the preceding Clauses 1-17, wherein a plurality of slots is disposed through a wall of the hinge structure, which plurality of slots communicate with the orifice, wherein a cylindrical tube element extends from the arm support structure, wherein at least a portion of the cylindrical tube element rotatably fits within the orifice, wherein at least one press button element is resiliently coupled to the cylindrical tube element, and wherein at least a portion of the press button element is configured to be reversibly disposed through any one of the plurality of slots such that the arm support structure is selectively rotatable about the axis.

Clause 19: The shoulder positioning device of any one of the preceding Clauses 1-18, wherein the plurality of slots comprises from about 10 to about 30 slots, wherein pairs of slots are disposed substantially diametrically opposite one another through the wall of the hinge structure.

Clause 20: The shoulder positioning device of any one of the preceding Clauses 1-19, wherein two press button elements are resiliently coupled to the cylindrical tube element, which press button elements are disposed substantially diametrically opposite one another on a circumference of the cylindrical tube element.

Clause 21: The shoulder positioning device of any one of the preceding Clauses 1-20, wherein the cylindrical tube element extends from the arm support structure via a platform component.

Clause 22: The shoulder positioning device of any one of the preceding Clauses 1-21, wherein the hinge structure comprises a substantially tubular shape.

Clause 23: The shoulder positioning device of any one of the preceding Clauses 1-22, wherein the plurality of slots provides angular adjustability to the arm support structure to effect external and/or internal rotation of the arm and a shoulder of the subject when the subject uses the shoulder positioning device.

Clause 24: The shoulder positioning device of any one of the preceding Clauses 1-23, wherein the press button element is rotatable at least between a locked position and an unlocked position.

Clause 25: The shoulder positioning device of any one of the preceding Clauses 1-24, wherein the press button element is resiliently coupled to the cylindrical tube element via at least one spring.

Clause 26: The shoulder positioning device of any one of the preceding Clauses 1-25, wherein the sliding mechanism comprises a slider element guide along which the slider element selectively moves.

Clause 27: The shoulder positioning device of any one of the preceding Clauses 1-26, wherein the slider element guide comprises a track element that extends along at least a portion of a length of the sliding mechanism and wherein the slider element comprises a track element receiving area that is configured to receive at least a segment of the track element.

Clause 28: The shoulder positioning device of any one of the preceding Clauses 1-27, wherein the sliding mechanism comprises a cover structure.

Clause 29: The shoulder positioning device of any one of the preceding Clauses 1-28, wherein the cover structure comprises at least one wall that limits movement of the slider element.

Clause 30: The shoulder positioning device of any one of the preceding Clauses 1-29, wherein the cover structure comprises two, three, four, or more walls.

Clause 31: The shoulder positioning device of any one of the preceding Clauses 1-30, wherein the sliding mechanism comprises a retaining mechanism that is configured to reversibly retain the slider element at a selected position along the radius.

Clause 32: The shoulder positioning device of any one of the preceding Clauses 1-31, wherein the sliding mechanism is adjustable for use on subjects with different arm lengths.

Clause 33: The shoulder positioning device of any one of the preceding Clauses 1-32, wherein the retaining mechanism comprises a body structure having a plurality of gripping surfaces.

Clause 34: The shoulder positioning device of any one of the preceding Clauses 1-33, wherein the retaining mechanism comprises a knob element that is configured to reversibly move the body structure such that at least one of the gripping surfaces grips the slider element to reversibly retain the slider element at the selected position along the radius.

Clause 35: The shoulder positioning device of any one of the preceding Clauses 1-34, wherein the knob element comprises a threaded cylindrical shaft component that is received by a corresponding threaded opening disposed through the wall of the cover structure, wherein the threaded cylindrical shaft component is configured to contact the body structure.

Clause 36: The shoulder positioning device of any one of the preceding Clauses 1-35, wherein the attachment mechanism comprises a vest that is configured to be worn around a torso portion of the subject.

Clause 37: The shoulder positioning device of any one of the preceding Clauses 1-36, wherein the attachment mechanism comprises a belt that is configured to be worn around a torso portion of the subject.

Clause 38: The shoulder positioning device of any one of the preceding Clauses 1-37, wherein the torso portion of the subject comprise a waist area of the subject.

Clause 39: The shoulder positioning device of any one of the preceding Clauses 1-38, wherein the attachment mechanism is operably connected or connectable to the base platform.

Clause 40: The shoulder positioning device of any one of the preceding Clauses 1-39, wherein the arm support structure is configured to substantially prevent an elbow of the subject from adducting when the subject uses the shoulder positioning device.

Clause 41: The shoulder positioning device of any one of the preceding Clauses 1-40, wherein the arm support structure is configured to adjust a degree of abduction of an upper arm of the subject relative to the torso of the subject when the subject uses the shoulder positioning device.

Clause 42: The shoulder positioning device of any one of the preceding Clauses 1-41, wherein the arm support structure comprises a tapered trough structure having an opening that is configured to receive at least the part of the arm of the subject when the subject uses the shoulder positioning device.

Clause 43: The shoulder positioning device of any one of the preceding Clauses 1-42, wherein the arm support structure is configured to be selectively rotated about the axis to effect external and/or internal rotation of the arm and a shoulder of the subject when the subject uses the shoulder positioning device.

Clause 44: The shoulder positioning device of any one of the preceding Clauses 1-43, wherein the arm support structure is configured to be selectively rotated about the axis to effect measurable amounts of external and/or internal rotation of the arm and a shoulder of the subject when the subject uses the shoulder positioning device.

Clause 45: The shoulder positioning device of any one of the preceding Clauses 1-44, wherein the arm support structure comprises one or more reversibly attachable retaining straps that are configured to retain at least a portion of the arm of the subject at least partially within the arm support structure when the subject uses the shoulder positioning device.

Clause 46: The shoulder positioning device of any one of the preceding Clauses 1-45, further comprising a covering structure disposed over at least a portion of the base frame.

Clause 47: A shoulder positioning device, comprising: a base frame comprising: a base platform comprising first and second surfaces; a first height adjustment mechanism operably connected to the base platform, wherein a height of the first height adjustment mechanism is configured to be selectively adjusted relative to the first surface of the base platform; a second height adjustment mechanism operably connected to the base platform, wherein a height of the second height adjustment mechanism is configured to be selectively adjusted relative to the second surface of the base platform; a main platform comprising first and second surfaces, wherein the main platform is operably connected to the first height adjustment mechanism; a sliding mechanism operably connected to the main platform, wherein the sliding mechanism comprises a slider element that is configured to selectively move radially along at least one radius; and an attachment mechanism operably connected or connectable to the base frame, wherein the attachment mechanism is configured to reversibly attach the base frame to a subject when the subject uses the shoulder positioning device; and, an arm support structure operably connected or connectable to the slider element of the sliding mechanism, wherein the arm support structure is configured to receive and support at least part of an arm of the subject when the subject uses the shoulder positioning device and wherein the arm support structure is configured to be selectively rotated about at least one axis.

Clause 48: A method of positioning a shoulder of a subject, the method comprising: attaching a shoulder positioning device to the subject using an attachment mechanism of the shoulder positioning device, wherein the shoulder positioning device comprises: a base frame comprising: a base platform comprising first and second surfaces; a sliding mechanism operably connected to the base frame, wherein the sliding mechanism comprises a slider element that is configured to selectively move radially along at least one radius; and the attachment mechanism operably connected to the base frame, wherein the attachment mechanism is configured to reversibly attach the base frame to the subject; and, an arm support structure operably connected to the slider element of the sliding mechanism, wherein the arm support structure is configured to receive and support at least part of an arm of the subject and wherein the arm support structure is configured to be selectively rotated about at least one axis; placing an arm of the subject in the arm support structure of the shoulder positioning device; moving the slider element of the sliding mechanism radially along the radius to a selected radial position relative to the subject; and rotating the arm support structure axially about the axis to a selected axial position relative to the subject, thereby positioning the shoulder of the subject.

Clause 49: The method of Clause 48, wherein the shoulder positioning device further comprises a first height adjustment mechanism operably connected to the base platform, wherein a height of the first height adjustment mechanism is configured to be selectively adjusted relative to the first surface of the base platform, and wherein the method further comprises adjusting the height of the first height adjustment mechanism relative to the first surface of the base platform.

Clause 50: The method of Clause 48 or Clause 49, wherein the shoulder positioning device further comprises a second height adjustment mechanism operably connected to the base platform, wherein a height of the second height adjustment mechanism is configured to be selectively adjusted relative to the second surface of the base platform, wherein the shoulder positioning device further comprises a main platform comprising first and second surfaces, wherein the main platform is operably connected to the first height adjustment mechanism, wherein the sliding mechanism is operably connected to the first surface of the main platform, and wherein the method further comprises adjusting the height of the second height adjustment mechanism relative to the second surface of the base platform.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A shoulder positioning device, comprising:
a base frame comprising:
a base platform comprising first and second surfaces;
a sliding mechanism operably connected to the base frame, wherein the sliding mechanism comprises a slider element that is configured to selectively move radially along at least one radius; and
an attachment mechanism operably connected or connectable to the base frame, wherein the attachment mechanism is configured to reversibly attach the base frame to a subject when the subject uses the shoulder positioning device; and,
an arm support structure operably connected or connectable to the slider element of the sliding mechanism, wherein the arm support structure is configured to receive and support at least part of an arm of the subject when the subject uses the shoulder positioning device and wherein the arm support structure is configured to be selectively rotated about at least one axis.

2. The shoulder positioning device of claim 1, further comprising a first height adjustment mechanism operably connected to the base platform, wherein a height of the first height adjustment mechanism is configured to be selectively adjusted relative to the first surface of the base platform.

3. The shoulder positioning device of claim 2, further comprising a second height adjustment mechanism operably connected to the base platform, wherein a height of the second height adjustment mechanism is configured to be selectively adjusted relative to the second surface of the base platform.

4. The shoulder positioning device of claim 2, further comprising a main platform comprising first and second surfaces, wherein the main platform is operably connected to the first height adjustment mechanism.

5. The shoulder positioning device of claim 2, wherein the first height adjustment mechanism is operably connected to the first surface of the base platform.

6. The shoulder positioning device of claim 2, wherein the height of the first height adjustment mechanism is configured to be selectively adjusted between about 10 mm and about 300 mm relative to the first surface of the base platform.

7. The shoulder positioning device of claim 2, wherein the first height adjustment mechanism comprises a set of telescopic legs.

8. The shoulder positioning device of claim 7, wherein the set of telescopic legs comprises one, two, three, four, five, or more telescopic legs.

9. The shoulder positioning device of claim 7, wherein a given telescopic leg of the first height adjustment mechanism comprises two, three, four, five, six, seven, eight, nine, or more telescoping sections.

10. The shoulder positioning device of claim 3, wherein the second height adjustment mechanism is operably connected to the second surface of the base platform.

11. The shoulder positioning device of claim 3, wherein the height of the second height adjustment mechanism is configured to be selectively adjusted between about 10 mm and about 300 mm relative to the second surface of the base platform.

12. The shoulder positioning device of claim 3, wherein the second height adjustment mechanism comprises a set of telescopic legs.

13. The shoulder positioning device of claim 12, wherein the set of telescopic legs comprises one, two, three, four, five, or more telescopic legs.

14. The shoulder positioning device of claim 12, wherein a given telescopic leg of the second height adjustment mechanism comprises two, three, four, five, six, seven, eight, nine, or more telescoping sections.

15. The shoulder positioning device of claim 3, wherein the first height adjustment mechanism and/or the second height adjustment mechanism are configured to effect abduction of an arm of a subject when the arm support structure receives the arm of the subject.

16. The shoulder positioning device of claim 1, wherein the sliding mechanism is operably connected to the first surface of the main platform.

17.-46. (canceled)

47. A shoulder positioning device, comprising:
a base frame comprising:
a base platform comprising first and second surfaces;
a first height adjustment mechanism operably connected to the base platform, wherein a height of the first height adjustment mechanism is configured to be selectively adjusted relative to the first surface of the base platform;
a second height adjustment mechanism operably connected to the base platform, wherein a height of the second height adjustment mechanism is configured to be selectively adjusted relative to the second surface of the base platform;
a main platform comprising first and second surfaces, wherein the main platform is operably connected to the first height adjustment mechanism;
a sliding mechanism operably connected to the main platform, wherein the sliding mechanism comprises a slider element that is configured to selectively move radially along at least one radius; and
an attachment mechanism operably connected or connectable to the base frame, wherein the attachment mechanism is configured to reversibly attach the base frame to a subject when the subject uses the shoulder positioning device; and,
an arm support structure operably connected or connectable to the slider element of the sliding mechanism, wherein the arm support structure is configured to receive and support at least part of an arm of the subject when the subject uses the shoulder positioning device and wherein the arm support structure is configured to be selectively rotated about at least one axis.

48. A method of positioning a shoulder of a subject, the method comprising:
attaching a shoulder positioning device to the subject using an attachment mechanism of the shoulder positioning device, wherein the shoulder positioning device comprises:
a base frame comprising:
a base platform comprising first and second surfaces;
a sliding mechanism operably connected to the base frame, wherein the sliding mechanism comprises a slider element that is configured to selectively move radially along at least one radius; and
the attachment mechanism operably connected to the base frame, wherein the attachment mechanism is configured to reversibly attach the base frame to the subject; and,
an arm support structure operably connected to the slider element of the sliding mechanism, wherein the arm support structure is configured to receive and support at least part of an arm of the subject and wherein the arm support structure is configured to be selectively rotated about at least one axis;
placing an arm of the subject in the arm support structure of the shoulder positioning device;
moving the slider element of the sliding mechanism radially along the radius to a selected radial position relative to the subject; and
rotating the arm support structure axially about the axis to a selected axial position relative to the subject, thereby positioning the shoulder of the subject.

49. The method of claim 48, wherein the shoulder positioning device further comprises a first height adjustment mechanism operably connected to the base platform, wherein a height of the first height adjustment mechanism is configured to be selectively adjusted relative to the first surface of the base platform, and wherein the method further comprises adjusting the height of the first height adjustment mechanism relative to the first surface of the base platform.

50. The method of claim 49, wherein the shoulder positioning device further comprises a second height adjustment mechanism operably connected to the base platform, wherein a height of the second height adjustment mechanism is configured to be selectively adjusted relative to the second surface of the base platform, wherein the shoulder positioning device further comprises a main platform comprising first and second surfaces, wherein the main platform is operably connected to the first height adjustment mechanism, wherein the sliding mechanism is operably connected to the first surface of the main platform, and wherein the method further comprises adjusting the height of the second height adjustment mechanism relative to the second surface of the base platform.
